(19) United States
(12) Patent Application Publication
(10) Pub. No.: US 2025/0279204 A1
(43) Pub. Date: Sep. 4, 2025

(54) X-RAY-BASED AI SOLUTIONS FROM MULTI-MODAL DATA

(71) Applicant: CARESTREAM HEALTH, INC., Rochester, NY (US)

(72) Inventors: Luca BOGONI, Philadelphia, PA (US); William J. SEHNERT, Fairport, NY (US)

(21) Appl. No.: 19/063,391

(22) Filed: Feb. 26, 2025

Related U.S. Application Data
(60) Provisional application No. 63/559,241, filed on Feb. 29, 2024.

(51) Int. Cl. G16H 50/20; G06T 7/00; G16H 50/70

(52) U.S. Cl. CPC G16H 50/20; G06T 7/0012; G16H 50/70; G06T 2207/10081; G06T 2207/10088; G06T 2207/10132; G06T 2207/20081; G06T 2207/20084

(57) ABSTRACT

A method to enhance the clinical value and role of x-ray imaging as part of the diagnostic and patient management chain using artificial intelligence (AI). The present invention adapts large collections of data acquired with modalities other than projection x-ray to enhance AI training that will enable x-ray imaging to more effectively be used in the full span of patient management from detection and diagnosis to management and therapy.

### CROSS REFERENCE TO RELATED APPLICATIONS

This application claims priority to U.S. Patent Application Ser. No. 63/559,241, filed Feb. 29, 2024, in the name of Bogoni et al., and entitled X-RAY-BASED AI SOLUTIONS FROM MULTI-MODAL DATA, which is hereby incorporated by reference herein in its entirety.

This application is related in certain respects to U.S. Pat. No. 8,705,828 filed Aug. 30, 2021, in the name of Yang et al., and entitled METHODS AND APPARATUS FOR SUPER RESOLUTION SCANNING FOR CBCT SYSTEM AND CONE-BEAM IMAGE RECONSTRUCTION; U.S. Pat. No. 11,158,050 filed Aug. 8, 2019, in the name of Huo et al., and entitled BONE SUPPRESSION FOR CHEST RADIOGRAPHS USING DEEP LEARNING; U.S. Pat. No. 11,478,213 filed Jan. 19, 2021, in the name of Wang et al., and entitled LOW DOSE DIGITAL TOMOSYNTHESIS SYSTEM AND METHOD USING ARTIFICIAL INTELLIGENCE; U.S. Pat. No. 11,553,891 filed May 7, 2020, in the name of Wang et al., and entitled AUTOMATIC RADIOGRAPHY EXPOSURE CONTROL USING RAPID PROBE EXPOSURE AND LEARNED SCENE ANALYSIS; U.S. Patent Application Publication No. US 2010/0121178 A1, filed Nov. 18, 2009, in the name of Krishnan et al., and entitled SYSTEMS AND METHODS FOR AUTOMATED DIAGNOSIS AND DECISION SUPPORT FOR BREAST IMAGING; U.S. Patent Application Publication No. US 2019/0357869 A1, filed Mar. 25, 2019, in the name of Madabhushi et al., and entitled PREDICTION OF RISK OF POST-ABLATION ATRIAL FIBRILLATION BASED ON RADIOGRAPHIC FEATURES OF PULMONARY VEIN MORPHOLOGY FROM CHEST IMAGING; and to U.S. Patent Application Publication No. US 2023/0306657 A1, filed Aug. 30, 2021, in the name of Sehnert et al., and entitled NOISE SUPPRESSION USING DEEP CONVOLUTIONAL NETWORKS; all of which are hereby incorporated by reference herein in their entirety.

### BACKGROUND OF THE INVENTION

The subject matter disclosed herein relates to a method to enhance the clinical value and role of x-ray imaging as part of the diagnostic and patient management chain using artificial intelligence (AI). The present invention adapts large collections of data acquired with other modalities to enhance AI solutions that will enable x-ray imaging to more effectively be used in the full span of patient management from detection and diagnosis to management and therapy.

One of the first imaging modalities used in diagnosing a patient is projection x-ray. However, while being extremely valuable and ubiquitous it presents with some limitations and often the patient is sent for differential diagnosis using CT. CT is then used not only as a modality to provide a differential diagnosis but may become the main imaging choice to manage the patient. Yet, many patients do not have access to CTs; furthermore, an early on-set of a disease or condition, which is latent in the x-ray and not appreciated or recognized by the radiologist in time, may lead to delays due to the inability of the radiologist to take timely action on the condition. As demonstrated in the US patents cited above, AI solutions have been providing great tools to detect some disease conditions so that the patient may be better managed. While in principle this will evolve to supply a veritable arsenal of tools to the physician, the ability to train AI modules, systems, algorithms and (neural) networks, which each may be referred to herein as “solutions,” depends on the availability of data (images, text annotations, and additional information). While for certain clinical conditions, such as detection of nodules (>8 mm) or bone fractures, pneumonia, pneumothorax, there are a plethora of x-ray data sets that can be used for training AI solutions. For a vast majority of clinical conditions projection x-ray imaging provides an inadequate or limited starting point. Once a manifestation of the disease is recognized or suspected, the patient may be transitioned to be managed using CT or another modality and projection x-ray imaging is no longer considered as the primary modality. As such, no follow up x-ray imaging data are acquired to enrich the pool of cases that could be used to train AI solutions.

The situation is more complex with certain diseases such as idiopathic pulmonary fibrosis (IPF). IPF represents a disease that is not easily diagnosed even in CT, as it is characterized as a diagnose of elimination. Thus, when all other potential diagnoses are ruled out, the patient is imputed with IPF. Telltale signs of the disease are present in the original x-ray image, but physicians are often not trained or able to recognize them. The limitations are also compounded by the level of experience of the physician and the prevalence of the disease in the general population. Less prevalent and harder to diagnose diseases linger untreated while the disease continues to progress.

Creating a training data repository that captures specific types of clinical conditions, acquired longitudinally using x-ray imaging, has inherent limitations especially when x-ray imaging is not designated as the standard of care. Thus, generating these training data collections requires deliberate and prospective studies explicitly designed to accrue a suitable number of cases. When the disease has a lower prevalence or, as in the case of IPF, is not easily recognized as a manifestation until much later in the disease progression, it becomes difficult or even impractical to create such a repository. When efforts are devoted toward this goal, they tend to be IRB driven, costly and lengthy especially when envisioning the task of collecting a large cohort of patients.

One embodiment of the present invention, described below, proposes to adapt large collections of data acquired with other modalities to enhance and enrich current x-ray data solutions that will enable x-ray imaging to effectively and efficiently be used in the full span of patient management from detection and diagnosis to management and therapy.

The discussion above is merely provided for general background information and is not intended to be used as an aid in determining the scope of the claimed subject matter.

### BRIEF DESCRIPTION OF THE INVENTION

A method to enhance the clinical value and role of x-ray imaging as part of the diagnostic and patient management chain using artificial intelligence (AI). The present invention adapts large collections of data acquired with modalities other than projection x-ray to enhance AI training that will enable x-ray imaging to more effectively be used in the full span of patient management from detection and diagnosis to management and therapy.

In one embodiment, image datasets are identified that are associated with a selected human disease condition, and x-ray projections are generated from the identified image datasets, which generated projections are then used to train an AI network to diagnose the selected human disease condition.

The premise of the present approach is predicated on two observations: (i) that there is a wealth of clinical data that has accrued over the past decades wherein patients are managed using modalities other than x-ray, even though the original detection of the onset of a disease process may be due to an original x-ray image acquisition; and (ii) it is possible to generate 2D x-ray projection images from CT volumetric data directly or following conversion to pseudo-CT or synthetic-CT data. While ultrasound imaging or x-ray imaging may be entry points in the diagnostic sequence, once CT, MRI, etc., are brought in for differential diagnosis these modalities tend to become the modalities of choice for continuing patient management. One embodiment of the present invention introduces a method to enhance the clinical value and role of x-ray imaging as part of the diagnostic and patient management workflow.

Expanding on the original observations, several aspects which are key to one embodiment of the present invention include a vast body of multi-dimensional data (CT, MRI, CT/PET) with many data points and associated diagnoses and clinical reports, not to mention metadata that pertain to the image itself (e.g., segmentation, measurements etc.); and that CT and x-ray technologies share many imaging characteristics yet have fundamental differences such as: CT images are quantified using Hounsfield units whereas x-ray imaging lacks the dynamic range, and CT imaging is volumetric whereas x-ray imaging is inherently 2D. X-ray imaging enjoys higher spatial resolution thus providing high quality and spatial acuity, although CT is certainly evolving also in this direction.

There is a large body of literature that has demonstrated the ability to perform cross-modality conversions to generate synthetic-CT (sCT), such as from MRI acquisitions. These synthetic-CTs are also referred to as pseudo-CT in the literature. This type of CT, generated from other modalities, may be referred to herein as sCT. While one embodiment of the present invention does not include these conversions, when a sCT may be available, synthetic-x-ray (s-x-ray) imaging projections can be generated thus expanding the available body of clinical imaging data for training AI. Additionally, the clinical contents and reports, and disease processes, may prove suitable for use to transfer diagnostic capabilities, and patient management capability primarily to x-ray imaging-a capability which, until now, may have been considered unlikely.

The summary descriptions above are not meant to describe individual separate embodiments whose elements are not interchangeable. In fact, many of the elements described as related to a particular embodiment can be used together with, and possibly interchanged with, elements of other described embodiments. Many changes and modifications may be made within the scope of the present invention without departing from the spirit thereof, and the invention includes all such modifications.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings below are intended to be drawn neither to any precise scale with respect to relative size, angular relationship, relative position, or timing relationship, nor to any combinational relationship with respect to interchangeability, substitution, or representation of a required implementation, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 is a flow diagram of exemplary CT (sCT) projection into projected sx-rays (marked with “P”) together with acquired x-rays and clinical lab data and known patient outcomes provided as input to train, test and validate AI algorithms yielding an AI engine that can operate on x-ray images and patient data to yield a predicted outcome;

FIG. 2 is an illustration of the method of FIG. 1 as an example (applied to thorax) expanded to include AI processing (6 and 7) as a means to provide structured information as well as results of specialized characterization;

FIG. 3 is a flow diagram of a further generalization of the method of FIG. 1, in this instance, as illustrated by label 8 in the figure, of providing actual CT data to the AI network;

FIG. 4 is a flow diagram of the AI engine, shown in FIG. 1-FIG. 3, processing data acquired directly using x-ray modality (a plurality of images (study) with associated clinical reports) to predict a patient outcome; and

FIG. 5 is a flow diagram of a more general version of an AI engine which predicts outcomes based not only on acquired x-ray images but also on all available data which may include prior CT acquired data, with possible projection x-ray images, AI processing of data by other 3^{rd} party solutions and, if available, data acquired by other modalities.

### DETAILED DESCRIPTION OF THE INVENTION

There are many AI solutions that can detect, diagnose, quantify, compute risk and confidence scores, provide decision support, selectively segment anatomy, disease processes or foreign objects. As used herein, description of the uses of CT data also applies to sCT data. In addition, a CT imaging modality may be referred to herein as the reference modality and x-ray imaging as the derived modality; the image data, clinical report, EMR, and ancillary information, etc., of the reference modality may be referred to as the reference data space, and the projection of image and related data into the derived x-ray modality maybe referred to as the derived data space.

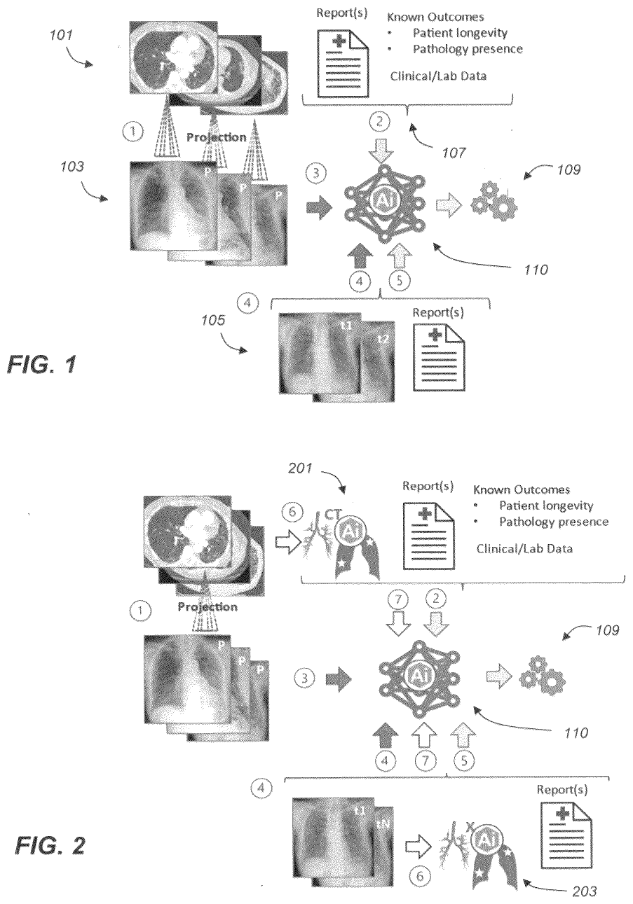

One embodiment of the present invention includes a method for training one or more AI solutions for the purpose of performing detection, diagnosis, quantification, patient management, and workflow improvement using one or more x-ray images, CT (sCT) images, and clinical information. As outlined in FIG. 1, one or more AI solutions 110 may be trained by incorporating derived data. This includes inputting collected data having volumetric imaging 101 as well as clinical or diagnostic information 107 acquired at a single or multiple timepoints, and generating derived x-ray images 103 from reference volumetric data 101. Developing, by training and validating, one or more AI solutions 110 using the generated derived x-ray images, to deliver a clinical resolution 109 such as to triage, detect, classify, quantify, or segment anatomical structures, disease processes or foreign objects. The AI solution 110 may be expanded, enriched, or enhanced to include x-ray images and clinical reports 105, acquired as part of the standard of care for the purpose of training the AI solution for triaging, screening, detecting, diagnosing, or managing a specific patient disease condition. This latter aspect guarantees that acquisition characteristics of x-ray devices are accounted for in the AI solution 110.

FIG. 2 outlines the same steps as in FIG. 1 but adds an embodiment of the present invention whereby the method is further enhanced by leveraging AI solutions (available in the market) that can post process CT images 201 as well as projection x-ray images 203 to provide a richer set of training inputs, illustrated as \textcircled{7}, to thereby architect one or more powerful AI solutions 110. As illustrated in FIG. 2, by applying one or more existing AI solutions or components developed for the reference data, these inputs may enhance, identify, detect, segment, quantify anatomical structures, disease processes or foreign objects in one or a longitudinal set of the reference data set. In addition, the reference data space might include confidence or risk scores. By projecting the results of the AI solutions from the reference data space to the derived data space, the AI solution 110 may be further optimized by including, in addition to images, selective x-ray projection images of a specific portion of the anatomy following segmentation, for example, an organ, a lesion, a disease process, into the derived data space.

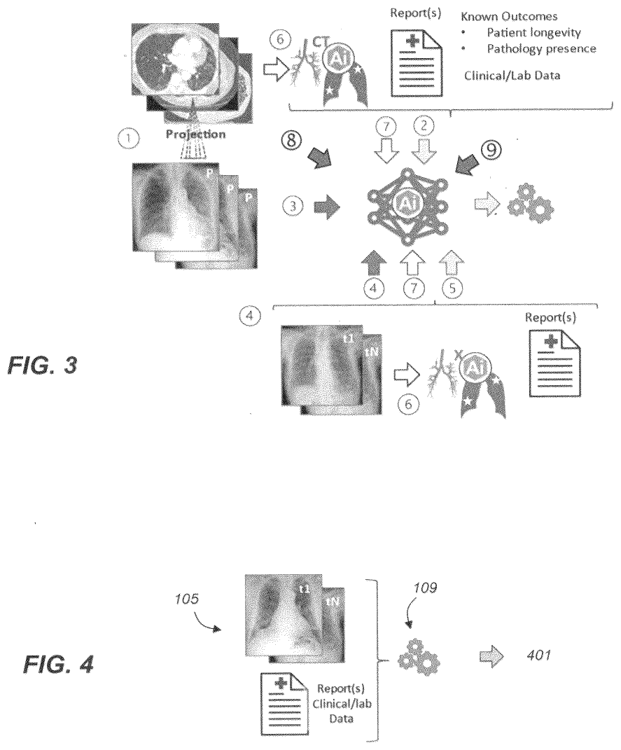

FIG. 3, which outlines the same steps as in FIGS. 1-2, further expands the outlined method of projecting the results of the AI solutions from the reference data space to the derived data space by enabling the construction of AI solutions that might incorporate not only x-ray images, whether s-x-ray or x-ray, but also images and data from various modalities such as actual CT data illustrated as @, and MRI, ultrasound (US), and non-imaging modalities such as electrocardiogram (EKG), spirometry, and blood pressure monitoring, illustrated as S.

Turning to FIG. 1, with a more detailed description, CT, or sCT, projection into projected sx-ray images, marked with the letter “P”, together with acquired x-ray images and reports and clinical/lab data and known patient outcome are provided as input 107 to train and validate an AI software solution 110 that can operate on x-ray images and, if available, patient data to yield a predicted outcome 109. As an example, the acquired (as well as projected) x-ray images 105 and data may be acquired at multiple time points for the same patient. The inclusion of x-ray acquired image data provides x-ray device characteristics which are not present in projected x-ray images 103 acquired from CT or sCT 101. The principal components of FIG. 1, \textcircled{1}, \textcircled{2}, \textcircled{3}, are used to derive an AI solution 110 from projection of CT (sCT) data and associated reports. The resulting AI solution 110 is further expanded, using components \textcircled{4}, \textcircled{5}, to include actual data and images 105 acquired by x-ray imaging. The images acquired from x-ray devices 105 may be a multiplicity of images acquired at different time points, and may include many more images from prior studies or may represent a dynamic sequence(s) of x-ray images from one or more studies. This method outlines an approach to train x-ray solutions 110 leveraging data from CT and sCT) for an x-ray modality that will empower physicians to manage patient conditions which are normally not detectable or quantifiable with x-ray imaging due to the limited availability of data or clinical annotations.

Turning to FIG. 2, the method can be expanded to include AI solutions 110 to operate on the reference data and produce outputs 109 which can range from anatomical segmentation masks, lesion segmentation as well as quantifications of these anatomies or disease processes. Additionally, the AI solutions 110 may process decision support clinical information such as: triaging, detection, and classification. In addition, the AI solution 110 may associate confidence scores or risk profiles/metrics following processing. FIG. 2 illustrates, by marking with stars, on the silhouette of lung masks obtained by an AI solution from a CT image, some detections or classification of lesions or disease processes. The white stars in segmented lungs illustrate some detections or characterization of pathologies/lesions. These AI outputs may, in turn, impact workflow management or patient management. AI solutions 110 may generate similar AI based outputs 109 from patient x-ray acquisitions 105. The collection of these AI characterizations and outputs can then be used to augment the training of AI solutions 110 that leverages both modalities. Thus, one embodiment includes AI processing as an input to provide structured training information as well as results of specialized characterization. One advantageous feature of the present invention, thus described, is that there might be only limited x-ray image data 105 available to train AI solutions, therefore, by projecting the CT reference data space into the derived data space, it is possible to obtain AI solutions 110 that provide information 109 directly from an x-ray input dataset.

Turning to FIG. 3, the methods disclosed herein are expanded to include not only projected CT (sCT) data but also the images themselves. In this instance, as illustrated by label \textcircled{8} in the figure, the method includes providing actual CT data 101 to the network 110. A further extension to a fully multi-modal approach can be envisioned, whereby other modalities, highlighted in the figure by \textcircled{9}, such as ultrasound, EKG, and MRI, may be provided as input to develop a more comprehensive AI solution 110. This would open the possibility to perform transfer learning on the original AI networks considering the enhancement to include x-ray images. This approach is advantageous, not only because the volumetric information is richer, given that the spatial relation of lesions, anatomy, disease processes are inherently lost in the projection process, but because the patient may have CT data that have been acquired as part of prior disease management or as part of the present disease process. For instance, a patient may undergo an x-ray examination as part of a screening, or upon suspicion of a disease. This may then be followed by additional imaging (CT or other imaging modality) for differential diagnosis; and the patient may then be managed using x-ray imaging. In such a scenario, which occur rather frequently, mix modality data may be made available for training AI networks. Being able to use this available data as described herein would yield more robust AI training and AI solutions.

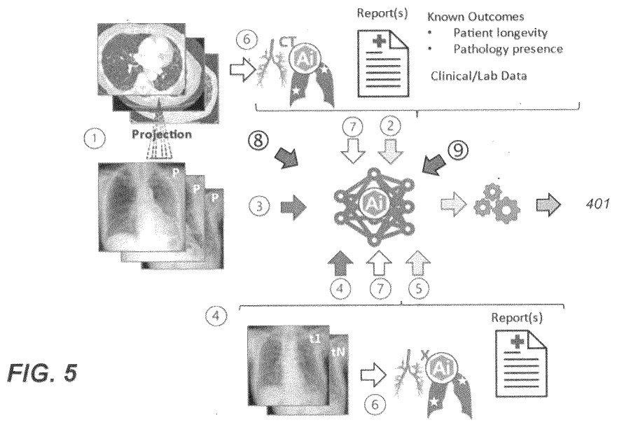

This latter extension provides further generalization by including other imaging and non-imaging modalities. This extension takes a more holistic approach considering the various data available and while it might prioritize patient management based on x-ray imaging, it would be able to include any ancillary information available to yield a stronger prognostication tool and lead to better outcome predictions. The derived data may be used to predict outcomes. FIG. 4 illustrates, using the method, taking as input actual acquired x-ray images to generate a prediction of outcome 401, or processing data acquired directly with an x-ray modality, i.e., a plurality of x-ray images with associated clinical reports, to predict a patient outcome 401. FIG. 4 shows providing one (t1) or a multiplicity of images (tN), which might have been acquired in previous sessions, or may illustrate incorporating a dynamic sequence of x-ray images from one or more studies. FIG. 5 illustrates a more general version of a method derived from using, as training input, a plurality of images to generate a predicted outcome, which predicts outcomes based not only on acquired x-ray images but also, based on all available data which may include prior CT acquired data, with possible projected x-ray images, AI processing of data by other 3^{rd} party solutions and, if available, data from other modalities.

As another example of the method disclosed herein, consider the context of improving the detection of lung nodules in x-ray imaging. To date, AI solutions which aid in the detection of lung nodules in x-ray are primarily capable of detecting solid nodules with a minimum diameter of 8 mm. Smaller or subsolid nodules are not part of lesions normally known to be detectable in x-ray imaging. On the other hand, there exist a vast amount of CT datasets with annotations of nodules >=3 mm. For instance, the National Lung Screening Trial (NLST) data collection, albeit with the last collection dating to 2009, has more than twenty five thousand multi-time point CT datasets. Another European lung nodule study (NELSON) also has collected, and continues to accrue, over thirty thousand patients, some of whom have been followed up for a decade. Leveraging datasets such as these can afford having annotated reference data which, upon projection into derived data, can be used to perform training both on smaller and different types of nodules. Having this capability available in the x-ray modality means that incidental smaller nodules may be identified as part of routine exams, detected in screening, or monitored as part of a follow up protocol, or rule-out metastatic cases.

Continuing the example of the lung nodule embodiment, by applying AI solutions to segment organs, such as lungs, in CT volumes, the resulting derived x-ray images may afford better visualization, enhancement, and improve and expedite the training of the AI solutions. The training can also include, as additional channels fed into the network, pairing of derived x-ray images of the full thorax as well as the derived x-ray projections of segmented lungs/parenchyma/thoracic cavity. These segmentations, for instance, would thus exclude all bony structures, and heart, thus enabling the AI networks to focus on the portion of the signal in the image which is derived from the organ in question. The projection of locations, segmentation, or other properties of the lesions of interest, e.g., nodules, as well as the confidence scores of various AI solutions applied to the reference CT data can be included for training and validation.

Additional embodiments aimed at addressing various disease or anatomical characterization are easily identifiable. For instance, large collections of patients mentioned above include other disease manifestations such as pulmonary emphysema, COPD, fibrosis and atelectasis. Other examples of large databases include diseases such as pulmonary embolism and COPD with patients who have been followed up for many years. While these are a few examples of large databases, the majority of CT (sCT) cases with clinical information are on PACS systems around the world; as such they present a vast source of potential derived x-ray images which could be used to develop and test convolutional neural networks. As noted previously, x-ray-based solutions may improve access to healthcare by increasing access and reducing cost. The embodiments above are illustrative of the method to transferring the diagnostic capabilities of AI solutions from one medical imaging domain, such as CT, to another medical imaging domain, such as x-ray, to supply clinical capabilities to a modality of choice.

As will be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.), or an embodiment combining software and hardware aspects that may all generally be referred to herein as a “service,” “circuit,” “circuitry,” “module,” and/or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Program code and/or executable instructions embodied on a computer readable medium may be transmitted using any appropriate medium, including but not limited to wireless, wireline, optical fiber cable, RF, etc., or any suitable combination of the foregoing. Computer program code for carrying out operations for aspects of the present invention may be written in any combination of one or more programming languages, including an object oriented programming language such as Java, Python, C++ or the like and conventional procedural programming languages, such as the “C” programming language or similar programming languages. The program code may execute entirely on the user's computer (device), partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method comprising:
identifying image datasets associated with a selected human disease condition;
generating x-ray projections from the identified image datasets; and
training an AI network on the generated x-ray projections to diagnose the selected human disease condition.

2. The method of claim 1, wherein the step of training the AI network includes using textual clinical reports.

3. The method of claim 1, wherein the step of identifying image datasets includes identifying CT, MRI, and ultrasound images.

4. The method of claim 1, wherein a selection of organs or anatomical regions is applied to the image datasets using masking or filtering of prior x-ray projection images.

5. The method of claim 3, wherein the CT datasets contain image data obtained by longitudinal acquisitions for the same patient over a period longer than twenty four hours.

6. The method of claim 5, wherein the step of training includes using current x-ray projection images acquired of the same patient as for the identified image datasets.

7. The method of claim 6, wherein the step of training includes detecting anatomical structures, disease processes or foreign objects.

8. The method of claim 7, wherein the step of training includes ranking the x-ray projections according to a selection of relevant conditions.

9. In a method of training an AI network using x-ray images, the improvement comprising including in an image training set images acquired from a modality distinct from x-ray imaging, and including textual annotation data.

10. The method of claim 9, wherein the modality distinct from x-ray imaging includes MRI imaging, ultrasound imaging and electrocardiogram data.
